(19) United States
(12) Patent Application Publication
(10) Pub. No.: US 2015/0359957 A1
(43) Pub. Date: Dec. 17, 2015

(54) AXIALLY SPLIT FOAM PERITONEAL DIALYSIS CATHETER DESIGN WITH FUNCTIONAL COATINGS

(71) Applicant: Cook Medical Technologies LLC, Bloomington, IN (US)

(72) Inventors: Kalub Hahne, West Lafayette, IN (US); Andrew P. Isch, West Lafayette, IN (US); Willie C. McRoy, West Lafayette, IN (US); Sara Marie Sherman, Lafayette, IN (US)

(21) Appl. No.: 14/723,854

(22) Filed: May 28, 2015

Related U.S. Application Data
(60) Provisional application No. 62/012,018, filed on Jun. 13, 2014.

(51) Int. Cl. A61M 1/28; A61L 29/08; A61L 29/16

(52) U.S. Cl. CPC A61M 1/285; A61L 29/16; A61L 29/08; A61M 2205/0205; A61M 2205/0238; A61L 2300/41; A61L 2300/416; A61L 2300/404; A61L 2300/204; A61L 2300/606

(57) ABSTRACT

The present disclosure provides a dialysis device for peritoneal dialysis. The device comprises a proximal tubular portion comprising a plurality of proximal lumens and a distal tubular portion comprising a plurality of independent tubes. Each independent tube has a distal lumen that is in fluid communication with one of the proximal lumens for delivery and retrieval of dialysis solution. The independent tubes are individually flexible and positionable with in the patient's body to aid in enhanced retainment of the device. The device also comprises one or more coatings. The coatings may be disposed on the entire device or a portion of the device to aid in reducing inflammation, irritation, leaking, or infection experienced during peritoneal dialysis.

### CROSS-REFERENCE TO RELATED APPLICATIONS

This application claims priority to U.S. Provisional Patent Application Ser. No. 62/012,018, filed Jun. 13, 2014, entitled “AXIALLY SPLIT FOAM PERITONEAL DIALYSIS CATHETER DESIGN WITH FUNCTIONAL COATINGS,” the entire contents of which are hereby incorporated by reference.

### BACKGROUND

1. Technical Field

The present disclosure relates to medical devices. More particularly, the disclosure relates to a device for peritoneal dialysis in the body of a patient.

2. Background Information

Peritoneal dialysis is a treatment for kidney disease in which fluid is delivered, dwells, and is flushed out of the patient's peritoneum on a periodic basis. A practitioner places a tube or catheter in the patient's abdominal cavity, and this tube delivers and retrieves the fluid, or dialysis solution.

Tube placement can be done using traditional surgery or minimally invasive procedures. Minimally invasive procedures include laparoscopy, using cameras or other visualization techniques, and percutaneous techniques, using a needle puncture to place the tube. Once the tube is in place, the patient can deliver and retrieve the dialysis solution at home. At home, the patient delivers solution, allows it to dwell in the body for a dwell time to exchange substances, and retrieves or expels the solution.

Common problems plaguing current catheters are placement and inflammation. Ideally, the catheter's distal end should be placed deep in the peritoneal cavity near the pubic bone. However, current catheters often shift once in place, which may cause the catheter to work incorrectly.

Additionally, peritoneal dialysis can inflame or infect the patient. For example, the dialysis solution can inflame the peritoneal cavity. In response, omentum tissue mounts an immune response. The omentum is a membrane layer or lining of the abdominal cavity. The immune response, or omental occlusion, causes the omentum tissue to grow inside of the catheter, clogging the distal region.

In addition to omental occlusion, inflammation, infection, irritation, and leaking can occur at the catheter's insertion site or all the way along the catheter as it tunnels into the body. There exists a need for an improved catheter that will solve the problems of maintaining proper positioning and inflammation.

### BRIEF SUMMARY

The present disclosure provides an improved device that delivers and retrieves dialysis solution for peritoneal dialysis in a patient's body. The device comprises a proximal tubular portion with a plurality of proximal lumens formed therethrough. The proximal tubular portion comprises a first end adapted to be disposed outside of the body, and extending distally to a first region. The first region connects with a second region at a connection point. The connection point may be at an exit or wound site in the body (e.g. patient's chest). The second region may be adapted to extend inside the body from the first region. Because the first end extends outside of the patient's body, the patient may remove it when the device is not in use. The second region may terminate in a second end.

The device further comprises a distal tubular portion having a proximal end being attached to the second end and extending to a plurality of distal tips. The distal tubular portion comprises a plurality of independent tubes. Preferably, the device has four independent tubes. Each independent tube may have a proximal longitudinal section attached to the second end and distally extending to a distal longitudinal section. The distal longitudinal section may form at least one aperture and may extend to one distal tip of the plurality of distal tips. Each independent tube may have a distal lumen formed through the proximal and distal longitudinal sections such that a first proximal lumen is in fluid communication with a first distal lumen.

Preferably, the at least one aperture comprises a plurality of apertures positioned circumferentially about or around each independent tube adjacent its respective distal tip. The distal tip is preferably open. Both the apertures and the distal tip aid in delivery and retrieval of dialysis solution.

The device further comprises a one or more coatings disposed on at least a part of the device. Preferably, the coating includes a therapeutic agent chosen from the group of an anti-inflammatory, antiproliferative, and antimicrobial agent. The antimicrobial agent may be antibacterial in one embodiment. In this manner, the independent tubes may be independently positionable from each other to allow selective delivery and retrieval of the dialysis solution in the body.

The proximal tubular portion may have a proximal cuff. In one embodiment, proximal cuff is a first cuff and a second cuff proximal the first cuff. Preferably, the first cuff may be disposed in the patient's rectus muscle. Preferably, the second cuff is disposed adjacent to the connection point of the first and second regions.

In one embodiment, the therapeutic agent is the anti-inflammatory agent and disposed on the device from the first cuff to the distal tip of each independent tube. Preferably, the anti-inflammatory agent is a non-steroidal anti-inflammatory (NSAID), selective cyclooxygenase-2 inhibitor (e.g. Nimusulide).

Additionally, the coating may be the antimicrobial agent. This coating, or any other coating, may be disposed entirely on the device. Alternatively, this coating, or any other coating, may be disposed on a part or portion of the device. Specifically, the antimicrobial agent may include Rifampin and Minocycline.

The proximal and distal tubular portions are preferably made of a material chosen from the group of silicone, polyurethane, and polytetrafluoroethylene (PTFE). Additionally, the material may be foam.

Optionally, each independent tube includes a stiffening member or a weight member to aid in enhanced retainment. The device comprises a fluoroscopic means, such as a barium strip, for visualization and enhanced retainment. The barium strip may be located in one or all of the device's lumens such that the proximal tubular portion and all independent tubes include a barium strip.

The device may be used according to a method of peritoneal dialysis for delivery and retrieval of dialysis solution. This method comprises (1) providing the device discussed herein; (2) introducing the device within the body; (3) delivering the dialysis solution through the device to the body; (4) allowing the dialysis solution to dwell for a predetermined time within the body; and (5) after allowing the dialysis solution to dwell, retrieving or expelling the dialysis solution with the device from the body of a patient.

In one embodiment, the step of allowing the dialysis solution to dwell comprises having each independent tube in fluid communication with the dialysis solution. Alternatively, the step of allowing the dialysis solution to dwell includes one independent tube being out of fluid communication with the dialysis solution after the step of delivering. To achieve this, the device may have the plurality of independent tubes having a first independent tube with a first length and a second independent tube with a second length, the first length being greater than the second length. Alternatively, the first length could be substantially the same as the second length.

### BRIEF DESCRIPTION OF THE DRAWINGS

FIG. 1 is an environmental view of a device in accordance with one embodiment of the present invention.

FIG. 2 is a side view of the device in accordance with one embodiment of the present invention.

FIG. 3 is a cross-sectional view of the device of FIG. 2.

FIG. 4 is a partial side view of the device of FIG. 2.

FIG. 5 is a partial side view of the device of FIG. 2.

FIG. 6 is a partial side view of the device of FIG. 2.

FIG. 7 is a partial side view of the device of FIG. 2.

FIG. 8 is a side view of the device of FIG. 2.

FIG. 9 is a method of peritoneal dialysis in accordance with one embodiment of the present invention.

### DETAILED DESCRIPTION OF THE DRAWINGS

The present disclosure will now be described more fully with reference to the accompanying figures, which show preferred embodiments. The accompanying figures are provided for general understanding of the structure of various embodiments. However, this disclosure may be embodied in many different forms. These figures should not be construed as limiting and they are not necessarily to scale.

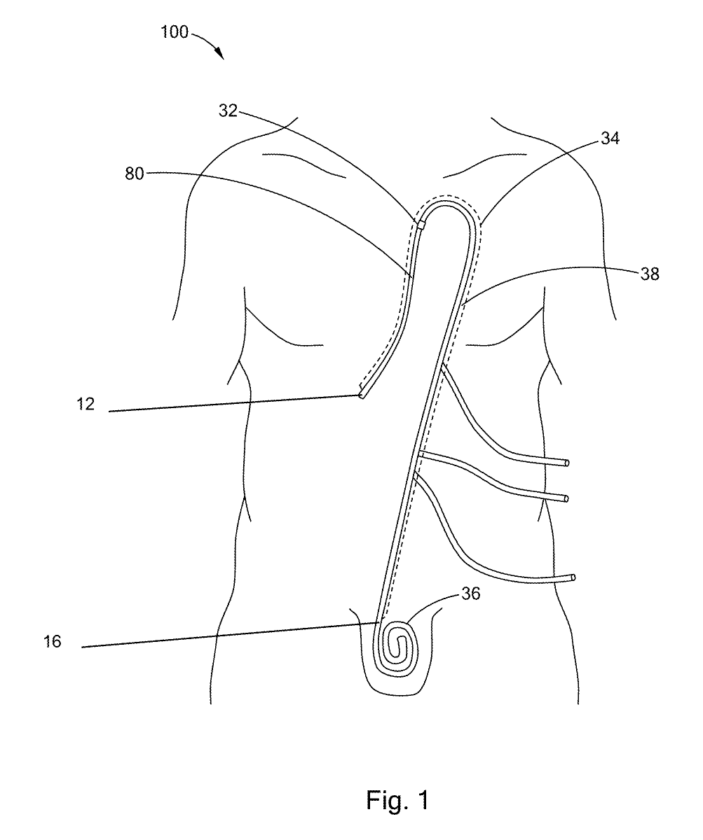

FIG. 1 is an environmental view 100 of one embodiment of the device in the body. A practitioner or physician introduces and positions the device in the body using known techniques, such as traditional surgery, laparoscopy, or percutaneous placement. The device may tunnel down from the insertion site (e.g. connection point 32), past the rib cage, to the public bone region. The device has two main portions: a proximal tubular portion 34 and a distal tubular portion 36. In FIG. 1, distal tubular portion 36 is coiled with one coil. It can also be uncoiled and may include further coils, as will be discussed further below.

The total length of the device may vary by its intended use and by individual patient. In a preferred embodiment, the entire device is approximately 40 to 50 centimeters (cm) in length. In a preferred embodiment, proximal tubular portion 34 is longer than distal tubular portion 36. Specifically, the proximal tubular portion is 30 centimeters long, and the distal tubular portion is 10 to 20 centimeters long.

The proximal tubular portion 34 has a first end 12 distally extending to a first region 80. The first region 80 may be adapted to be disposed outside of the body, and may be removable as the patient needs. The first region 80 extends distally to a second region 38 adapted to extend inside the body from a connection point or exit site 32. The second region 38 extends down through the ribs to a second end 16. The second end 16 connects with the distal tubular portion 36.

When placing the device, the practitioner creates exit site 32, which is a wound that heals around the device in the patient's body. The distal tubular portion 36 continues down to the patient's pubic bone. The pubic bone region is ideal for delivery and retrieval of dialysis solution with the device.

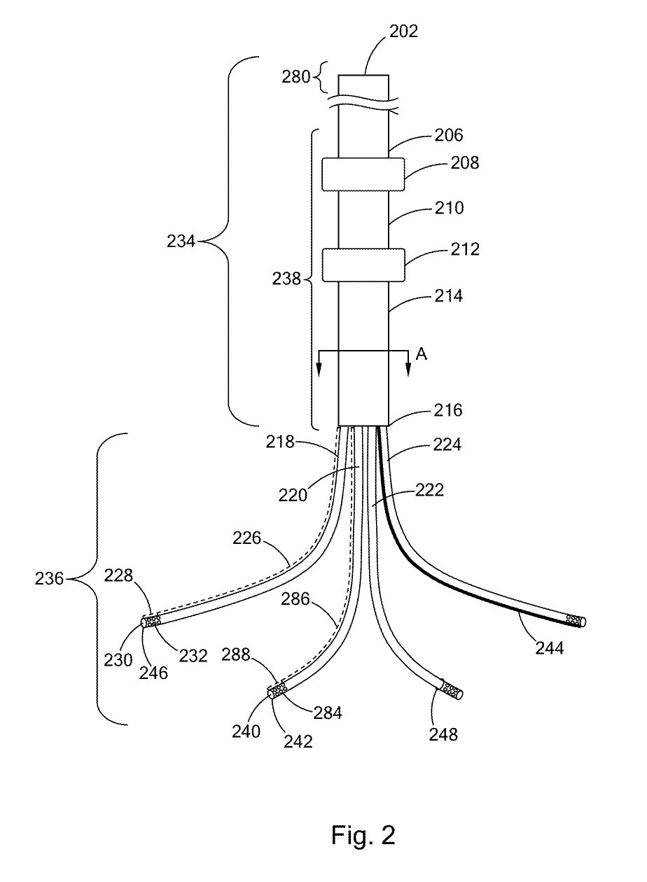

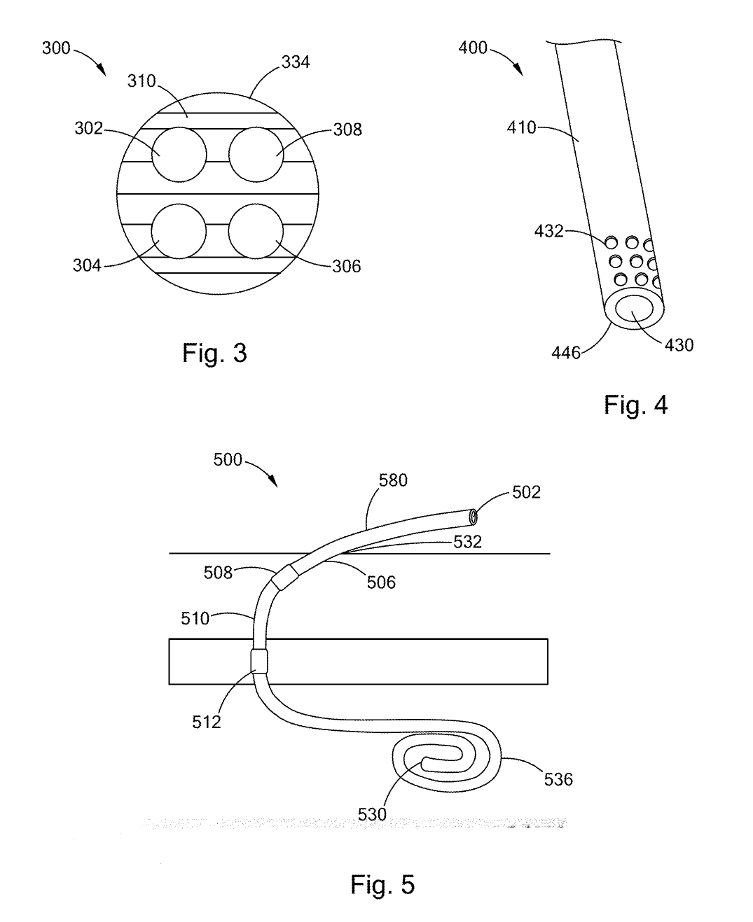

FIG. 2 is a side view of one embodiment of the device. The first region 280 of the proximal tubular portion 234 connects to the second region 238. Second region 238 connects to distal tubular portion 236 at second end 216. The proximal tubular portion comprises a plurality of proximal lumens 202 formed therethrough, shown in further detail in cross-section A in FIG. 3. In a preferred embodiment, the proximal tubular portion is nonporous.

Second region 238 is divided into segments 206, 210, and 214. These segments are separated by a proximal cuff. The proximal cuff may be a first cuff 212 and second cuff 208 proximal the first cuff 212. These cuffs aid in enhanced retainment of the device in the patient's body. The first cuff 212 is positioned and configured to be disposed in the patient's rectus muscle. The second cuff 208 is optional for enhanced retainment, and is positioned and configured to be disposed adjacent to the connection point or exit site (FIG. 1, 32). “Adjacent” means that the second cuff is located next to, near, or in close proximity with the connection of the first and second regions. The second cuff 208 may aid in wound healing at the exit site.

The material for the cuffs can vary as necessary based on its intended use and individual patient. In a preferred embodiment, both cuffs are Dacron® cuffs. The second cuff 208 can be placed adjacent to the exit site, separated from the exit site by segment 206. Segment 210 separates the second cuff 208 from the first cuff 212, and this segment may be sufficiently long enough to properly dispose the cuffs in the body. The second region 238 may terminate at the second end 216.

The distal tubular portion 236 contains a proximal end being attached to the second end 216 and extending to a plurality if distal tips (e.g. 246, 242). The distal tubular portion 236 contains a plurality of independent tubes. In FIG. 2, four independent tubes are shown (218, 220, 222, 224). In the preferred embodiment, each distal tip is open (exposing distal lumens 230 and 240, respectively) to aid in delivery and retrieval of dialysis solution.

A skilled artisan will understand that more or less independent tubes can be used as desired for proper placement and maintaining positioning of the device. The greater the number of independent tubes, the better the device will maintain its position in the pubic bone region. While the independent tubes connect at second end 216, they all maintain individual flexibility and orientation. This individual flexibility allows for each independent tube to be placed at different locations within the patient's body. The independent tubes can all extend in different directions, acting to anchor the device in the patient's body. Increased tension on the proximal tubular portion, through several independent tubes, may result in the device maintaining its position in the body.

Additionally, the practitioner can position each independent tube for maximum delivery and retrieval of dialysis solution. In this embodiment, each independent tube delivers and retrieves dialysis solution because each has a distal tip positioned near the pubic bone and in fluid communication with dialysis solution after it has been delivered to the patient. Optionally in this embodiment, all of the independent tubes can be the same length

Each independent tube has a proximal longitudinal section (e.g. 226) attached to the second end 216 and distally extending to a distal longitudinal section (e.g. 228). For example in FIG. 2, independent tubes 218 and 220 have proximal longitudinal sections 226 and 286, and distal longitudinal sections 228 and 288, respectively. The proximal longitudinal sections are in fluid communication with the distal longitudinal sections, through proximal and distal lumens. The proximal longitudinal sections 226 and 286 may be nonporous, while the distal longitudinal sections 228 and 288 may be porous. As discussed above, the proximal lumens 202 containing a plurality of proximal lumens (FIG. 3, 302, 308, 304, 306) in fluid communication with the distal lumens. For example, proximal lumen 302 may be in fluid communication with distal lumen 230 (FIG. 2). In addition, a first proximal lumen 304 may be in fluid communication with a first distal lumen 240.

Each distal longitudinal section (e.g. 228, 288) may form at least one aperture (e.g. 232, 284). Further, the distal longitudinal sections (e.g. 228, 288) may extend to a distal tip (e.g. 246, 242, respectively). Although these features are shown on only two independent tubes, it will be understood that each independent tube may have any or all of the features discussed herein. In a preferred embodiment, one aperture is a plurality of apertures being formed circumferentially around each independent tube adjacent to its respective distal tip, as depicted in FIG. 2. “Adjacent” means that the apertures are located next to, near, or in close proximity with the distal tip so that they may be close enough to deliver and retrieve dialysis solution near the treatment site.

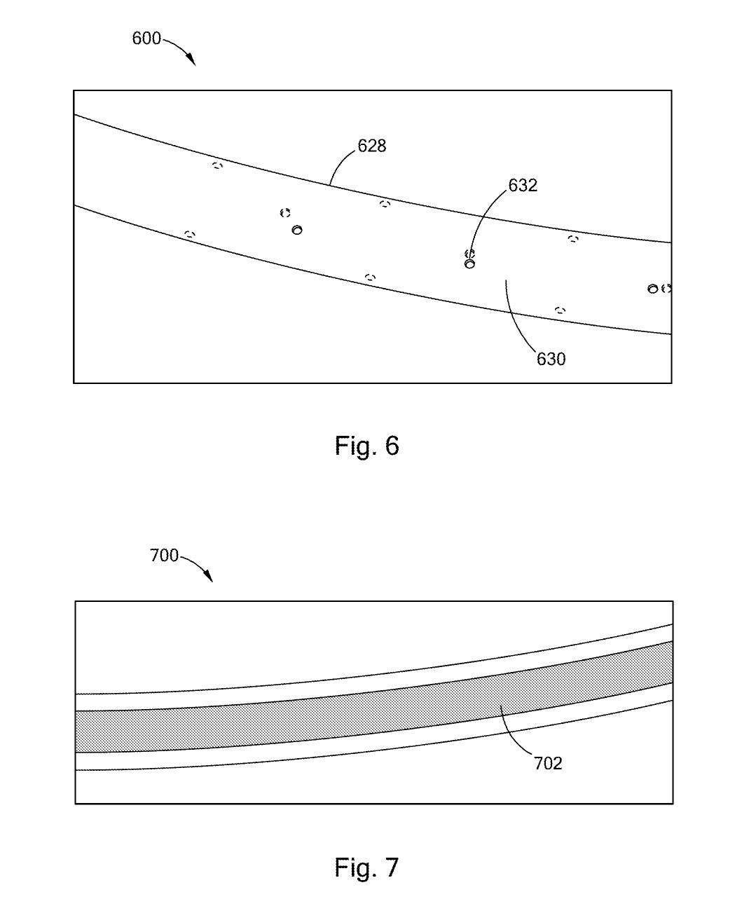

Each aperture may have a size and shape suitable for its intended use and individual patient. In a preferred embodiment, the apertures have a circular shape. The apertures aid in delivery and retrieval of dialysis solution. Further details of the apertures are discussed with FIG. 6.

In one aspect, at least one of the independent tubes may be positioned to be out of fluid communication with the dialysis solution after its delivery. These independent tubes may be out of fluid communication as a function of their position or length. Optionally, the independent tubes may vary in length so that the plurality of independent tubes has a first independent tube 218 with a first length and a second independent tube 220 with a second length such that the first length is greater than the second length. Alternatively, the first length may be about the same as the second length. “About” or “substantially” mean that the quantities (e.g. lengths) are within 10%, preferably within 5%, more preferably within 1% of each other. These independent tubes of different lengths or sizes may all deliver and retrieve dialysis solution if they are all placed in fluid communication with the dialysis solution.

However, if the second independent tube 220 with the second length is not placed in fluid communication with the dialysis solution once it has been delivered to the patient's body, it can deliver but not retrieve dialysis solution. In this embodiment, all of the independent tubes can deliver dialysis solution, but not all of the independent tubes will be placed to retrieve it from the peritoneal cavity.

The diameter of each independent tube can vary between independent tubes and along the length of the tubes, based on intended use and individual patient. In a preferred embodiment, the diameter of each independent tube is smaller than the diameter of the proximal tubular portion.

As with length and diameter, the shape of the independent tubes may vary by intended application and individual patient. In one embodiment, the independent tubes are in an uncoiled shape, as shown in FIG. 2. Alternatively, they can have a coiled shape, as shown in FIGS. 1 and 5. Retaining the independent tubes in an uncoiled shape can be aided by stiffening member 244, shown in FIG. 2. Alternatively, the distal tip may be weighted with a weight member 248. The weight member 248 and the stiffening member 244 may aid in enhanced retainment of the device (in any independent tube).

FIG. 3 is a cross-sectional view 300 of the device from line A. In cross-section 300, luminal space 202 is split into a plurality of proximal lumens. In one embodiment, four different proximal lumens are formed longitudinally down the proximal tubular portion 334: proximal lumens 302, 304, 306, and 308. These proximal lumens run longitudinally along proximal tubular portion 334, and are in fluid communication with the distal tubular portion (not shown here).

Cross-section 300 also shows material 310, which makes up the proximal tubular portion 334. Material 310 can be any suitable material for peritoneal dialysis. In a preferred embodiment, material 310 will be one of silicone, polyurethane, or polytetrafluoroethylene (PTFE). Additionally, material 310 may be foam. Material 310 may be used to form the distal tubular portion as well.

Material 310 can also vary along the length, diameter, or by part of the device. In addition, material 310 can also have varying degrees of hardness based on its intended use and individual patient. In one embodiment, material 310 is 50A durometer silicone foam. Alternatively, material 310 is 80A durometer silicone foam. A harder material may lead to reduced omental occlusion. Additionally, a harder material may be desirable in overweight or obese patients, or in female versus male patients.

FIG. 4 is a partial side view 400 of the distal tubular portion. The distal tubular portion contains aperture 432 adjacent to the distal tip 446. Distal lumen 430 runs through the distal tubular portion. In this embodiment, material 410 of the distal tubular portion is foam PTFE.

FIG. 5 is a side view 500 of one embodiment. FIG. 5 shows only one independent tube in distal tubular portion 536 for simplicity. In this embodiment, the distal tubular portion 536 is in a coiled configuration. Proximal lumen 502 may be formed longitudinally through first region 580, exit site 532, segment 506 before the second cuff 508, segment 510 separating the second cuff 508 from the first cuff 512, and is in fluid communication with distal lumen 530. This demonstrates that one proximal lumen 502 may be in fluid communication with one distal lumen 530.

FIG. 6 is a blown-up, side view 600 of one embodiment of the apertures. Aperture 632 in distal longitudinal section 628 is circular in shape and evenly spaced from adjacent or immediately neighboring apertures. In this embodiment, aperture 632 may be representative of all apertures in the distal longitudinal section 628. In one embodiment, the distal longitudinal section contains one hundred (100) circular apertures, evenly spaced from each other and located adjacent to the distal tip around or about the circumference of the distal tubular portion. In one embodiment, the size of one aperture is 3 French, providing fluid delivery from the proximal and distal tubular portions (e.g. through proximal lumen 630), out the apertures to the body cavity.

FIG. 7 is a partial side view 700 of a barium strip 702 disposed in or along a lumen. Barium strip 702 can be used in fluoroscopic techniques to visualize, position, and reposition the device. In FIG. 7, barium strip 702 is only shown in a portion of the device, but it will be understood by a skilled artisan that it would extend throughout all lumens, in the proximal tubular portion and all independent tubes.

The device may also contain one or more coatings, being disposed on a part of the device. In one embodiment, a coating can be used to prevent or alleviate inflammation and infection. The coating may have a therapeutic agent of one of an anti-infammatory, antiproliferative, and antimicrobial agent, or some combination of all of the above as desired.

Any coating or agent may cover the entire device or be located on a portion of the device. Methods of coating the device include those that are well known to one skilled in the art. Some examples include impregnating or embedding the coating in the device's material, dipping, spraying, or painting the device and allowing it to dry.

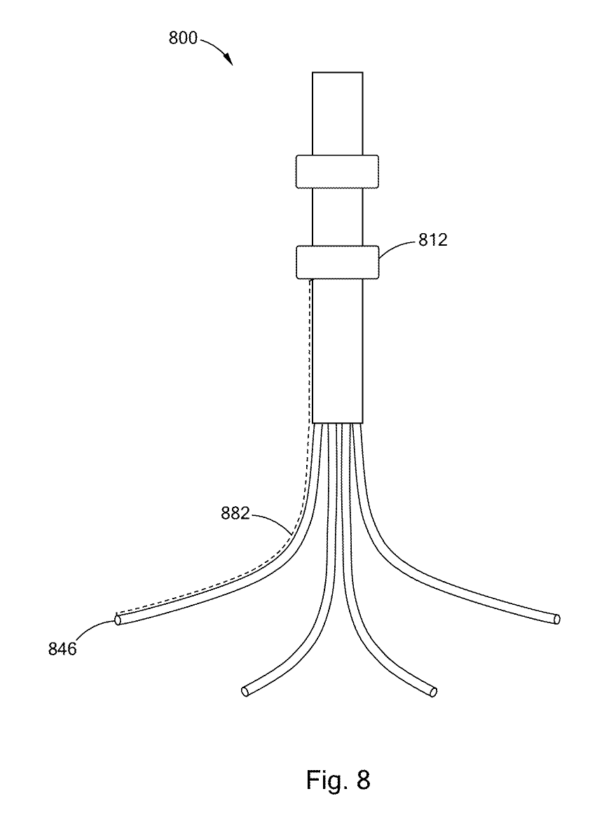

FIG. 8 shows a side view 800 of one embodiment of a coating 882. In this embodiment, the coating contains a therapeutic agent being an anti-inflammatory agent disposed on the device from the first cuff 812 to all distal tips, such as distal tip 846. Even though coating 882 is depicted with a dotted line, this line simply represents that the coating and therapeutic agent may be disposed entirely or partially on an outer surface generally in the region depicted by the line. In a preferred embodiment, the anti-inflammatory agent would be a non-steroidal anti-inflammatory (NSAID), selective cyclooxygenase-2 inhibitor. In a preferred embodiment, the anti-inflammatory agent is Nimesulide.

While the anti-inflammatory agent could reduce inflammation, other agents could treat bacterial or microbial infection. In addition, an antiproliferative agent could reduce endothelialization and cellular proliferation onto and around the device. In one embodiment, the antimicrobial agent is disposed on the entire device. Alternatively, the antimicrobial agent is disposed selectively on the first region of the device, extending outside of the patient's body. In one embodiment, the antimicrobial agent includes a mixture of Rifampin and Minocycline. A skilled artisan will understand that the device may be coated with agents even if the patient is prescribed related systemic treatments.

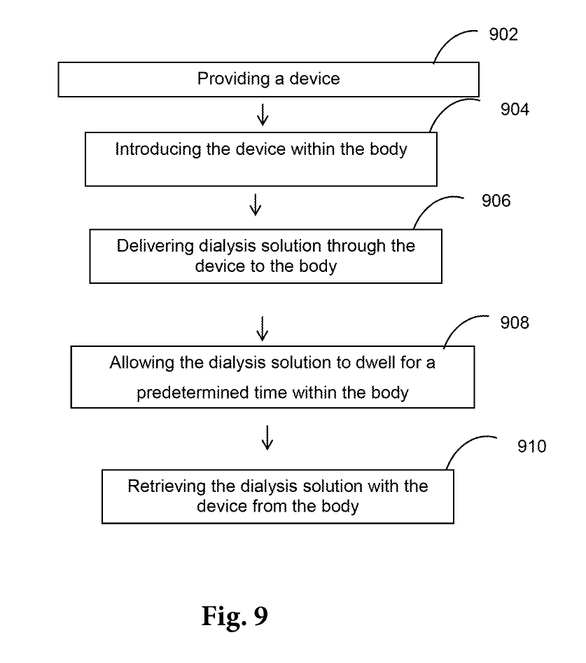

FIG. 9 illustrates a method of peritoneal dialysis with the device. In step 902, the practitioner may provide the device. The device may have any or all of the features or aspects discussed herein. In step 904, the practitioner may introduce the device within the body. In step 906, the practitioner or patient may deliver dialysis solution through the device to the body or the abdominal cavity. As discussed above, delivery and retrieval of dialysis solution may occur through all of the independent tubes.

Alternatively, if the plurality of independent tubes comprises a first independent tube being longer than a second independent tube, the second independent tube may not reach the peritoneal cavity near the public bone. While the first independent tube is sized and positioned to be in fluid communication with and reach the dialysis solution in the patient's body, the second independent tube delivers and does not retrieve dialysis solution because it is sized and positioned to be out of fluid communication with the dialysis solution in the patient's body. Retrieval of dialysis solution only occurs through independent tubes in fluid communication with the dialysis solution in the patient's body.

In step 908, the practitioner or patient may allow the dialysis solution to dwell in the body for a dwell time determined by the practitioner. In step 910, the practitioner or patient may retrieve the dialysis solution through the device after the appropriate dwell time.

It should be understood that above disclosure of the device and method of its use is merely exemplary of this peritoneal dialysis system, and not intended to limit the scope of the disclosure. Other parts, assemblies, and methods may be used without falling beyond the scope and spirit of the present disclosure, as defined in the following claims.

1. A device for performing peritoneal dialysis with a dialysis solution in a body of a patient, the device comprising:
a proximal tubular portion comprising a plurality of proximal lumens formed therethrough, the proximal tubular portion having a first end distally extending to a first region, the first region adapted to be disposed outside of the body, the first region extending distally to a second region adapted to extend inside the body, the second region terminating in a second end;
a distal tubular portion having a proximal end being attached to the second end and extending to a plurality of distal tips, the distal tubular portion comprising a plurality of independent tubes, each independent tube having a proximal longitudinal section attached to the second end and distally extending to a distal longitudinal section, the distal longitudinal section forming at least one aperture and extending to one distal tip of the plurality of distal tips, each independent tube having a distal lumen formed through the proximal and distal longitudinal sections; and
a coating disposed on a part of the device, the coating comprising a therapeutic agent chosen from the group of an anti-inflammatory, antiproliferative, and antimicrobial agent, the independent tubes being independently positionable from each other to allow selective delivery and retrieval of the dialysis solution in the body.

2. The device of claim 1 wherein a first proximal lumen is in fluid communication with a first distal lumen.

3. The device of claim 1 wherein the plurality of independent tubes comprises four independent tubes.

4. The device of claim 1 wherein the at least one aperture comprises a plurality of apertures, the plurality of apertures being formed circumferentially around each independent tube adjacent to its respective distal tip.

5. The device of claim 1 wherein each independent tube comprises a stiffening member for enhanced retainment in the body.

6. The device of claim 1 wherein the proximal tubular portion and each independent tube comprises a barium strip for enhanced retainment in the body.

7. The device of claim 1 wherein each independent tube comprises a weight member for enhanced retainment in the body.

8. The device of claim 1 wherein the proximal tubular portion comprises a proximal cuff for enhanced retainment in the body.

9. The device of claim 8 wherein the proximal cuff is a first cuff and a second cuff proximal the first cuff, the second cuff being disposed adjacent to a connection point between the first and second regions.

10. The device of claim 9 wherein the coating is disposed on the device from the first cuff to the distal tip of each independent tube, the therapeutic agent being the anti-inflammatory agent.

11. The device of claim 10 wherein the anti-inflammatory agent is a non-steroidal anti-inflammatory, selective cyclooxygenase-2 inhibitor.

12. The device of claim 11 wherein the non-steroidal anti-inflammatory, selective cyclooxygenase-2 inhibitor is Nimusulide.

13. The device of claim 1 wherein the therapeutic agent being the antimicrobial agent is disposed on the entire device.

14. The device of claim 13 wherein the antimicrobial agent comprises Rifampin and Minocycline.

15. The device of claim 1 wherein the proximal and distal tubular portions are made of a material, the material chosen from the group of silicone, polyurethane, and polytetrafluoroethylene.

16. The device of claim 1 wherein the plurality of independent tubes comprises a first independent tube with a first length and a second independent tube with a second length, the first length being greater than the second length.

17. The device of claim 1 wherein the plurality of independent tubes comprises a first independent tube with a first length and a second independent tube with a second length, the first length being the substantially same as the second length.

18. A method of peritoneal dialysis for delivery and retrieval of a dialysis solution in a body of a patient, the method comprising:
providing a device, the device comprising;
a proximal tubular portion comprising a plurality of proximal lumens formed therethrough, the proximal tubular portion having a first end distally extending to a first region, the first region adapted to be disposed outside of the body, the first region extending distally to a second region adapted to extend inside the body, the second region terminating in a second end;
a distal tubular portion having a proximal end being attached to the second end and extending to a plurality of distal tips, the distal tubular portion comprising a plurality of independent tubes, each independent tube having a proximal longitudinal section attached to the second end and distally extending to a distal longitudinal section, the distal longitudinal section forming at least one aperture and extending to one distal tip of the plurality of distal tips, each independent tube having a distal lumen formed through the proximal and distal longitudinal sections; and
a coating disposed on a part of the device, the coating comprising a therapeutic agent chosen from the group of an anti-inflammatory, antiproliferative, and antimicrobial agent, the independent tubes being independently positionable from each other to allow selective delivery and retrieval of the dialysis solution in the body;
introducing the device within the body;
delivering the dialysis solution through the device to the body;
allowing the dialysis solution to dwell for a predetermined time within the body; and
after allowing the dialysis solution to dwell for a predetermined time, retrieving the dialysis solution with the device from the body.

19. The method of claim 18 wherein the step of allowing the dialysis solution to dwell comprises having each independent tube in fluid communication with the dialysis solution.

20. The method of claim 18 wherein the step of allowing the dialysis solution to dwell comprises having at least one independent tube being out of fluid communication with the dialysis solution.
